(19) United States
(12) Patent Application Publication
(10) Pub. No.: US 2025/0182531 A1
(43) Pub. Date: Jun. 5, 2025

(54) LIVING BODY DETECTION SUPPORT APPARATUS, FACE AUTHENTICATION APPARATUS, LIVING BODY DETECTION SUPPORT METHOD, FACE AUTHENTICATION METHOD, PROGRAM, AND RECORDING MEDIUM

(71) Applicant: NEC Solution Innovators, Ltd., Koto-ku, Tokyo (JP)

(72) Inventor: Kazuhide UMEDA, Tokyo (JP)

(73) Assignee: NEC Solution Innovators, Ltd., Koto-ku, Tokyo (JP)

(21) Appl. No.: 18/843,371

(22) PCT Filed: Jan. 23, 2023
(86) PCT No.: PCT/JP2023/001856

§ 371 Date: Sep. 3, 2024

(30) Foreign Application Priority Data
Mar. 15, 2022 (JP) 2022-039941

(51) Int. Cl. G06V 40/40; G06V 40/16; G06V 40/20; G06V 40/60

(52) U.S. Cl. CPC G06V 40/45; G06V 40/172; G06V 40/20; G06V 40/67

(57) ABSTRACT

An apparatus comprising: processor and storage, wherein the storage is configured to: store determination criteria information, wherein, the determination criteria information is information in which types of lip motion and lip information for each of the types of lip motion are associated with each other, the processor is configured to: output lip motion instruction information, wherein the lip motion instruction information includes information instructing a given type of lip motion, acquire lip image information including a mouth of a subject person, acquire lip information of the subject person from the lip image information, determine whether the subject person performed the instructed lip motion, on the basis of the determination criteria information, the lip motion instruction information, and the lip information of the subject person, and determine that the subject person is a living body when it is determined that the subject person performed the instructed lip motion.

### INCORPORATION BY REFERENCE

This application is based upon and claims the benefit of priority from Japanese patent application No. 2022-039941 filed on Mar. 15, 2022, the disclosure of which is incorporated herein in its entirety by reference.

### TECHNICAL FIELD

The present invention relates to a living body detection support apparatus, a face authentication apparatus, a living body detection support method, a face authentication method, a program, and a recording medium.

### BACKGROUND ART

In recent years, face authentication technology has been used to perform personal authentication in various scenes.

Although face authentication has many advantages such as that it can be executed in a non-contact manner, it also has a risk for being broken through by deception using a face image or a video of a subject person. For this reason, a face authentication apparatus provided with a function of liveness determination (also referred to as living body determination or living body detection, for example) which is performed by detecting a color tone of a face or a facial motion of a subject person, is known as a technique for preventing spoofing with such deception (Patent Literature 1)

### CITATION LIST

### Patent Literature

Patent literature 1: JP 2020-184331A

### SUMMARY

### Technical Problem

However, since spoofing methods are increasing year by year, a new living body detection method is required.

An example object of the invention is to provide a living body detection support apparatus which realizes living body detection.

### Solution to Problem

A living body detection support apparatus according to an example aspect of the invention includes: a determination criteria information storage part; an instruction information output part; an image information acquisition part; a lip information acquisition part; a lip motion determination part; and a living body determination part, wherein the determination criteria information storage part stores determination criteria information, the determination criteria information is information in which types of lip motion and lip information for each of the types of lip motion are associated with each other, the instruction information output part outputs lip motion instruction information, the lip motion instruction information includes information instructing a given type of lip motion, the image information acquisition part acquires lip image information including a mouth of a subject person, the lip information acquisition part acquires lip information of the subject person from the lip image information, the lip motion determination part determines whether the subject person performed the instructed lip motion, on the basis of the determination criteria information, the lip motion instruction information, and the lip information of the subject person, and the living body determination part determines that the subject person is a living body when it is determined that the subject person performed the instructed lip motion.

A face authentication apparatus according to an example aspect of the invention includes: a living body detection part; a face image acquisition part; and an authentication part, wherein the living body detection part is the living body detection support apparatus according to an example aspect of the invention, and executes living body detection of an authentication subject person, the face image acquisition part acquires a face image of the authentication subject person, and the authentication part executes face authentication of the authentication subject person on the basis of the face image of the authentication subject person and authentication criteria information.

A living body detection support method according to an example aspect of the invention includes: outputting instruction information; acquiring image information; acquiring lip information; determining lip motion; and determining living body, wherein the outputting instruction information outputs lip motion instruction information, the lip motion instruction information includes information instructing a given type of lip motion, the acquiring image information acquires lip image information including a mouth of a subject person, the acquiring lip information acquires the lip information of the subject person from the lip image information, the determining lip motion determines whether the subject person performed the instructed lip motion, on the basis of determination criteria information, the lip motion instruction information, and the lip information of the subject person, the determination criteria information is information in which types of lip motion and lip information for each of the types of lip motion are associated with each other, and the determining living body determines that the subject person is a living body, when it is determined that the subject person performed the instructed lip motion.

A face authentication method according to an example aspect of the invention includes: detecting a living body; acquiring a face image; and authenticating, wherein the detecting a living body is the living detection support method according to an example aspect of the invention, and executes living body detection of an authentication subject person, the acquiring a face image acquires the face image of the authentication subject person, and the authenticating executes face authentication of the authentication subject person on the basis of the face image of the authentication subject person and authentication criteria information.

A first program for causing a computer to execute following processes, according to an example aspect of the invention includes: an instruction information output process; an image information acquisition process; a lip information acquisition process; a lip motion determination process; and a living body determination process, wherein the instruction information output process outputs lip motion instruction information, the lip motion instruction information includes information instructing a given type of lip motion, the image information acquisition process acquires lip image information including a mouth of a subject person, the lip information acquisition process acquires lip information of the subject person from the lip image information, the lip motion determination process determines whether the subject person performed the instructed lip motion, on the basis of determination criteria information, the lip motion instruction information, and the lip information of the subject person, the determination criteria information is information in which types of lip motion and lip information for each of the types of lip motion are associated with each other, and the living body determination process determines that the subject person is a living body when it is determined that the subject person performed the instructed lip motion.

A second program for causing a computer to execute following processes, according to an example aspect of the invention includes: a living body detection process; a face image acquisition process; and an authentication process, wherein the living body detection process is executed by the first program, and executes living body detection of an authentication subject person, the face image acquisition process acquires a face image of the authentication subject person, and the authentication process executes face authentication of the authentication subject person, on the basis of the face image of the authentication subject person and authentication criteria information.

### Advantageous Effects of Invention

An example advantage according to the invention is that a living body detection can be achieved.

### BRIEF DESCRIPTION OF DRAWINGS

FIG. 1 is a block diagram illustrating an exemplary configuration of a living body detection support apparatus of the first example embodiment.

FIG. 2 is a block diagram illustrating an exemplary hardware configuration of the living body detection support apparatus of the first example embodiment.

FIG. 3 is a flow chart showing an exemplary processing in the living body detection support apparatus of the first example embodiment.

FIG. 4 is a block diagram showing an exemplary configuration of a face authentication apparatus of the second example embodiment.

FIG. 5 is a block diagram showing an exemplary hardware configuration of the face authentication apparatus of the second example embodiment.

FIG. 6 is a flow chart showing an exemplary processing in the face authentication apparatus of the second example embodiment.

### EXAMPLE EMBODIMENTS

The example embodiments of the present invention are described below with reference to the drawings. It is to be noted, however, that the present invention is by no means limited or restricted by the following example embodiments. In the following drawings, identical parts are indicated with identical reference signs. Regarding the descriptions of the example embodiments, reference can be made to one another unless otherwise stated. Furthermore, the configurations of the example embodiments can be combined unless otherwise stated.

### First Example Embodiment

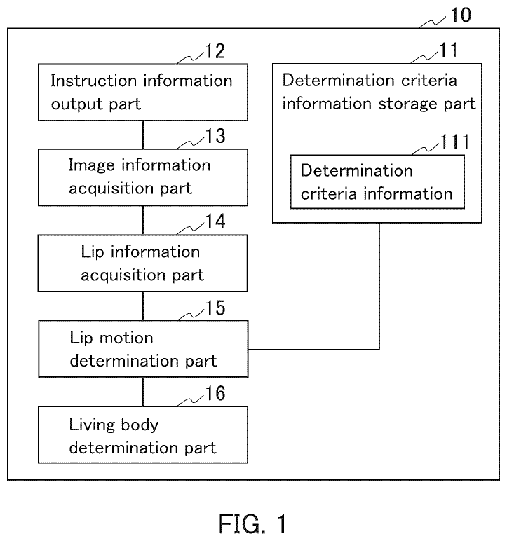

FIG. 1 is a block diagram illustrating an exemplary configuration of a living body detection support apparatus 10 of the present example embodiment. As shown in FIG. 1, the apparatus 10 includes a determination criteria information storage part 11, an instruction information output part 12, an image information acquisition part 13, a lip information acquisition part 14, a lip motion determination part 15, and a living body determination part 16. As shown in FIG. 1, the determination criteria information storage part 11 includes determination criteria information 111. The determination criteria information 111 will be described later.

The apparatus 10 may be, for example, one apparatus including the aforementioned parts, or may be an apparatus to which the respective parts are connectable via a communication line network. Further, the apparatus 10 is connectable to an external device described later, via the communication line network. The communication line network is not particularly limited and may be a known network, and may be wired or wireless, for example. Examples of the communication line network include an internet line, WWW (World Wide Web), a telephone line, LAN (Local Area Network), SAN (Storage Area Network), DTN (Delay Tolerant Networking), LPWA (Low Power Wide Area), and L5G (local 5G). Examples of wireless communication include WI-FI (registered trademark), BLUETOOTH (registered trademark), local 5G, and LPWA. The wireless communication may be a form where apparatuses directly communicate with each other (Ad-Hoc communication), infrastructure communication, indirect communication via an access point, or the like. The apparatus 10 may be built in a server as a system, for example. The apparatus 10 may also be, for example, a personal computer (PC, e.g., a desktop PC and a laptop), a smartphone, a tablet terminal, or the like with the program of the present invention installed therein. The apparatus 10 may be in a form of cloud computing, edge computing, or the like such that at least one of the parts is on a server and the rest of the parts are on a terminal, for example.

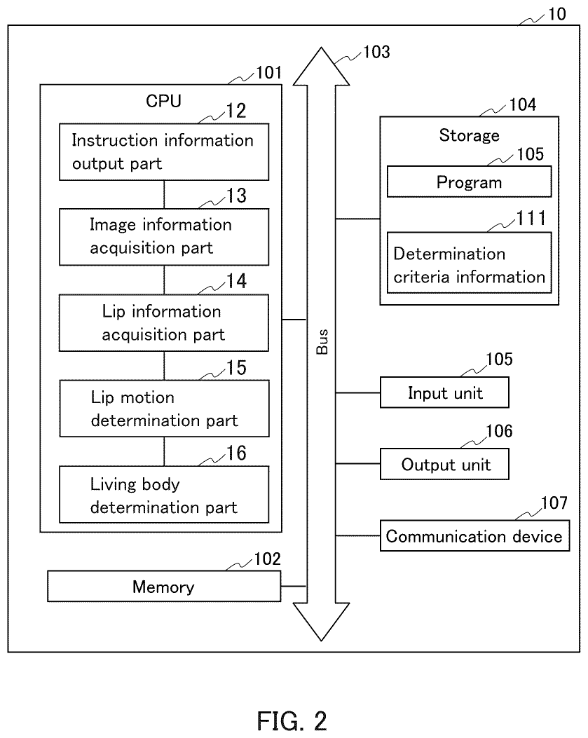

FIG. 2 is a block diagram illustrating an exemplary hardware configuration of the apparatus 10. The apparatus 10 includes, for example, a CPU 101, a memory 102, a bus 103, a storage 104, an input unit 106, an output unit 107, a communication device 108, and the like. The units of the apparatus 10 are connected to each other by their respective interfaces (I/F), via the bus 103.

The CPU 101 operates in cooperation with other configurations with a controller (a system controller, a I/O controller, or the like), and is responsible for control of the entire apparatus 10. In the apparatus 10, the CPU 101 executes, for example, a program 105 of the present invention or other programs, and reads and writes various data. Specifically, for example, the CPU 101 functions as an instruction information output part 12, an image information acquisition part 13, a lip information acquisition part 14, a lip motion determination part 15, and a living body determination part 16. The apparatus 10 includes the CPU as an arithmetic unit, but may include another arithmetic unit such as a GPU (Graphics Processing Unit) and an APU (Accelerated Processing Unit), or a combination of these and the CPU.

The bus 103 is connectable to an external device, for example. Examples of the external device include a face authentication apparatus such as a face authentication apparatus of the present invention to be described later, an external storage (external data base, etc.), a printer, an external input unit, an external display, and an external imaging device. The apparatus 10 can be, for example, connected to an external network (the communication line network) through the communication device 108 connected to the bus 103, and can be connected to other devices via the external network.

The memory 102 may be, for example, a main memory (main storage). When the CPU 101 executes processing, for example, the memory 102 reads various operation programs such as the program of the present invention stored in the storage 104 to be described later, and the CPU 101 receives the data from the memory 102 to execute the program. The main memory is, for example, a random-access memory (RAM). The memory 102 may also be, for example, a ROM (Read-Only Memory).

The storage 104 is also referred to as a so-called auxiliary storage with respect to the main memory (main storage), for example. As mentioned above, operation programs including the program of the present invention are stored in the storage 104. The storage 104 may be, for example, a combination of a recording medium and a drive for performing reading and writing on a recording medium. The recording medium is not particularly limited and may be a built-in type or an external type, and examples thereof include HD (Hard Disk), CD-ROM, CD-R, CD-RW, MO, DVD, a flash memory, and a memory card. The storage 104 may be, for example, a hard disk drive (HDD) in which a recording medium and a drive are integrated, or a solid state drive (SSD). The storage 104 functions as a determination criteria information storage part 11, for example.

In the apparatus 10, the memory 102 and the storage 104 can also store various information such as log information, information acquired from an external data base (not shown) or an external device, information generated by the apparatus 10, and information used when the apparatus 10 executes processing. In this case, the memory 102 and the storage 104 may store at least a piece of information selected from the group consisting of, for example, determination criteria information 111, lip motion instruction information, lip image information, lip information, a result of the determination performed by the living body determination part 16, and the like, which will be described later. It should be noted that, at least a piece of the information may be, for example, stored in an external server other than the memory 102 and the storage 104, or may be dispersedly stored in a plurality of terminals using a blockchain technique or the like.

The apparatus 10 further includes an input unit 106 and an output unit 107, for example. Examples of the input unit 106 include a pointing device such as a touch panel, a track pad, and a mouse; a keyboard; an imaging device such as a camera and a scanner; a card reader such as an IC card reader and a magnetic card reader; and an audio inputting unit such as a microphone. Examples of the output unit 107 include a display such as an LED display and a liquid crystal display; an audio output unit such as a speaker; and a printer. In the first example embodiment, the input unit 106 and the output unit 107 are configured separately, but the input unit 106 and the output unit 107 may be configured integrally like a touch panel display.

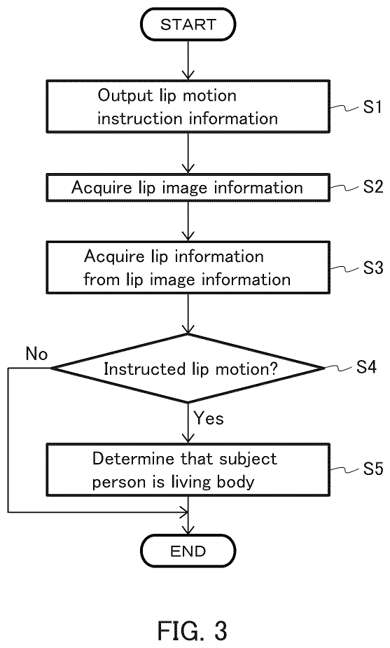

Next, an example of the living body detection support method according to the present example embodiment will be described with reference to the flow chart of FIG. 3. The living body detection support method according to the present example embodiment may be performed, for example, as follows using the living body detection support apparatus 10 of FIG. 1 or FIG. 2. Note that, the living body detection support method according to the present example embodiment is not limited to the use of the living body detection support apparatus 10 of FIG. 1 or FIG. 2.

Firstly, prior to the processing by the apparatus 10, a determination criteria information storage part 11 stores determination criteria information 111. The determination criteria information 111 is information serving as criteria for determining whether a subject person moved his/her mouth as instructed, in which types of lip motion and lip information for each of the types of lip motion are associated with each other. The type of lip motion is not particularly limited, and examples thereof include an opening or closing state of a mouth, phonation of a given character, and formation of a given lip shape. The given character is not particularly limited, and may be, for example, a consonant, a vowel, an alphabet, or the like, and is preferred to be a vowel. Further, the given character may be, for example, a character consisting of one sound (e.g., “A”, “I”, “U”, “E”, “O”, etc.), characters consisting of two or more sounds (e.g., “AI”, “IO”, “AIU”, “IUE”, “AEO”, etc.), or a combination of these. The lip information is, for example, information for representing a state of a lip, and specific examples thereof include lip coordinate information indicating a position of a lip in image information, lip shape information indicating a lip shape, and lip motion information indicating lip motion. In the determination criteria information 111, a combination of the type of lip motion and the lip information to be associated is not particularly limited, and for example, any combination can be utilized. Specific examples of the determination criteria information 111 include information in which the type of lip motion (phonation of various vowels) and the lip coordinate information for each type of vowels are associated with each other. Described in the following is an example assuming that the determination criteria information 111 is information in which the phonation of various vowels and the lip coordinate information for each type of vowels are associated with each other, but the present invention is by no means limited thereto.

Next, an instruction information output part 12 outputs lip motion instruction information (S1, outputting instruction information). The lip motion instruction information includes, for example, information instructing a given type of lip motion. The number of the types of lip motion included in the lip motion instruction information is not particularly limited, and may be, for example, one or two or more, and is preferred to be two or more for the improvement of accuracy of the living body detection, for example. For example, the instruction information output part 12 decides a type of lip motion which is randomly picked up from the types of lip motion stored as the determination criteria information 111, and outputs information prompting a subject person to perform the lip motion of the decided type, as the lip motion instruction information. Specific examples of the lip motion instruction information include information instructing an opening or closing state of a mouth, information instructing phonation of a given character, information instructing formation of a given lip shape, and a combination of these. If the lip motion instruction information includes the information instructing phonation of a given character, for example, the lip motion instruction information is preferred to include information instructing phonation of two or more characters. A format for outputting the lip motion instruction information by the instruction information output part 12 is not particularly limited, and may be for example, text information, voice information, or a combination of these. Specifically, the instruction information output part 12 may, for example, output voice information of a sentence such as “Read the characters displayed on the screen” by the output unit 107 connected via the bus 103, and display given characters such as “AIU” on the screen, so as to output information instructing phonation of “AIU” as the lip motion instruction information.

Next, an image information acquisition part 13 acquires information of a lip image including a mouth of a subject person (S2, acquiring image information). The lip image information is not particularly limited as long as being an image imaging a lip of a subject person, and may be, for example, a face image of a subject person. The lip image information may be, for example, a still image such as a photograph, or a moving image. The image information acquisition part 13 may acquire the image continuously or intermittently, for example. In the latter case, the image information acquisition part 13 may acquire the image at every given time elapsed, or at any timing. When the lip motion instruction information includes information instructing phonation of a given character, for example, the lip image information can be acquired by detecting the start of the phonation by the subject person with a sound collecting device such as a microphone. The image information acquisition part 13 may, for example, acquire the lip image information by imaging around the lip (e.g., a face) of the subject person by the imaging device, which is the input unit 106, or may acquire the lip image information from an external imaging device by the communication device 108 via the communication line network. When the apparatus 10 is capable of communicating with an external face authentication apparatus, for example, a face image imaged by the face authentication apparatus for face authentication may be acquired as the lip image information, from the face authentication apparatus. The image information acquisition part 13 may, for example, store the acquired lip image information in the memory 102 or the storage 104.

Next, a lip information acquisition part 14 acquires lip information of a subject person from the lip image information (S3, acquiring lip information). For example, the lip information acquisition part 14 can extract a characteristic point of a lip part of a subject person from the lip image information and analyze the characteristic point using an image analyzing technique to acquire the lip information. The lip information is, for example, as previously described.

Next, a lip motion determination part 15 determines whether a subject person performed the instructed lip motion, on the basis of the determination criteria information, the lip motion instruction information, and lip information of the subject person (S4, determining lip motion). For example, the lip motion determination part 15 specifies lip information at the time when the type of lip motion included in the lip motion instruction information outputted in S is performed on the basis of the determination criteria information, and executes matching of the specified lip information and the lip information of the subject person acquired in S3 to determine whether the subject person performed the instructed lip motion. Specifically, the lip motion determination part 15 firstly specifies reference lip coordinate information on the basis of the determination criteria information and the lip motion instruction information, for example. The reference lip coordinate information is, for example, lip coordinate information at the time when the lip motion included in the lip motion instruction information outputted in S1 is performed. Then, the lip motion determination part 15 determines whether the reference lip coordinate information matches with the lip coordinate information of the subject person, to determine whether the subject person performed the lip operation instructed in S1, for example.

When it is determined that the subject person performed the instructed lip motion (S4, Yes), then a living body determination part 16 determines that the subject person is a living body (S5, determining a living body), and terminates the processing (END). When it is determined that the subject person has not performed the instructed lip motion in S4 (S4, No), the apparatus 10 may terminate the processing (END), or execute any process among S1 to S4 again, or output the effect that the authentication has failed, for example.

The living body detection support apparatus of the present example embodiment can achieve a new living body determination (liveness determination) using lip information of a subject person, for example. Specifically, the living body detection support apparatus of the present example embodiment is capable of, for example, executing matching of randomly decided lip motion and lip motion of a subject person to perform living body detection of the subject person. Since instructing phonation of two or more randomly decided characters as the lip motion instruction information and executing matching of the lip coordinate information at the time of the phonation of the plurality of character makes it difficult to prepare moving image information in advance, spoofing can be prevented more effectively.

### Second Example Embodiment

The second example embodiment is an example of the face authentication apparatus of the present invention.

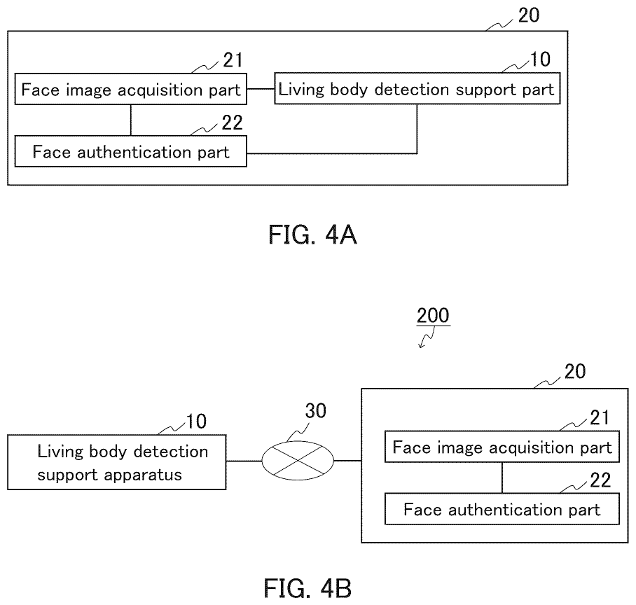

A face authentication apparatus of the present example embodiment will be described with reference to FIGS. 4A and 4B. FIGS. 4A and 4B are block diagrams illustrating an exemplary configuration of the face authentication apparatus of the present example embodiment. As shown in FIG. 4A, a face authentication apparatus 20 of the present example embodiment includes, for example, a face image acquisition part 21, a face authentication part 22, and a living body detection support part 10. The living body detection support part 10 is, for example, the same as the living body detection support apparatus 10 of the first example embodiment, and the explanation thereof can be incorporated. For this reason, in the following description, the descriptions of the “living body detection support part 10” and the “living body detection support apparatus 10” can be used interchangeably, for example. Note that, although a detail configuration of the living body detection support part 10 (living body detection support apparatus 10) is not shown in FIGS. 4A and 4B, the living body detection support part 10 includes the same configuration as the living body detection support apparatus 10 of the first example embodiment. The face authentication apparatus 20 may include, for example, a storage part which is not shown.

The face authentication apparatus 20 may be, for example, one apparatus including the aforementioned parts, or may be an apparatus to which the respective parts are connectable via a communication line network. Further, the face authentication apparatus 20 is connectable to an external device to be described later via the communication line network. The communication line network may be as described above, for example. The face authentication apparatus 20 may be built in a server as a system, for example. The face authentication apparatus 20 may be, for example, a personal computer (PC, e.g., a desktop PC or a laptop), a smartphone, a tablet terminal, or the like with the program of the present invention installed therein. The face authentication apparatus 20 may be in a form of, for example, cloud computing, edge computing, or the like, such that at least one of the parts is on a server and the rest of the parts are on a terminal. A specific example is, as shown in FIG. 4B, a form of a face authentication system 200 where the face authentication apparatus 20 including the face image acquisition part 21 and the face authentication part 22, and the living body detection support apparatus 10 of the first example embodiment as a living body detection support part 10 are connected via a communication line network 30.

In the face authentication apparatus of the second example embodiment, for example, the face image acquisition part 21 acquires a face image of the authentication subject person, and the face authentication part 22 executes face authentication of the authentication subject person on the basis of the face image of the authentication subject person and the authentication reference information.

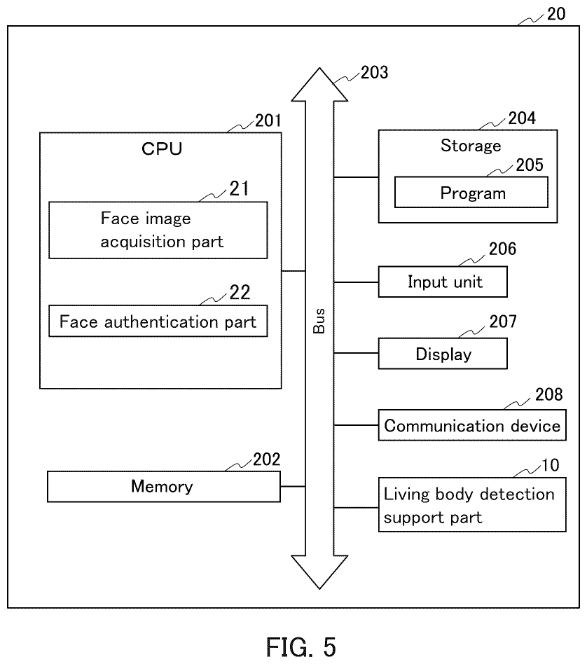

FIG. 5 illustrates a block diagram of an exemplary hardware configuration of the face authentication apparatus 20. As shown in FIG. 5, the face authentication apparatus 20 includes, for example, a CPU 201, a memory 202, a bus 203, a storage 204, an input unit 206, a display 207, a communication device 208, and the like. The description of each configuration of the face authentication apparatus 20 can incorporate the description of each configuration of the living body detection support apparatus 10. The parts of the face authentication apparatus 20 are connected to each other via the bus 203 through their respective interfaces (I/F). In the face authentication apparatus 20, the CPU 201 functions as the face image acquisition part 21, and the face authentication part 22. The face authentication apparatus 20 in FIG. 5 is in an exemplary form where the living body detection support part 10 (living body detection support apparatus 10) is connected to the bus 203, but the present invention is not limited thereto. In the face authentication apparatus 20, for example, the living body detection support part 10 may be connected by the communication device 208 via the communication line network 30, and the program for executing the respective processes of the living body detection support method according to the first example embodiment may be stored in the storage 204 or the memory 202, and the CPU 201 may read and execute the program to function as the living body detection support part 10.

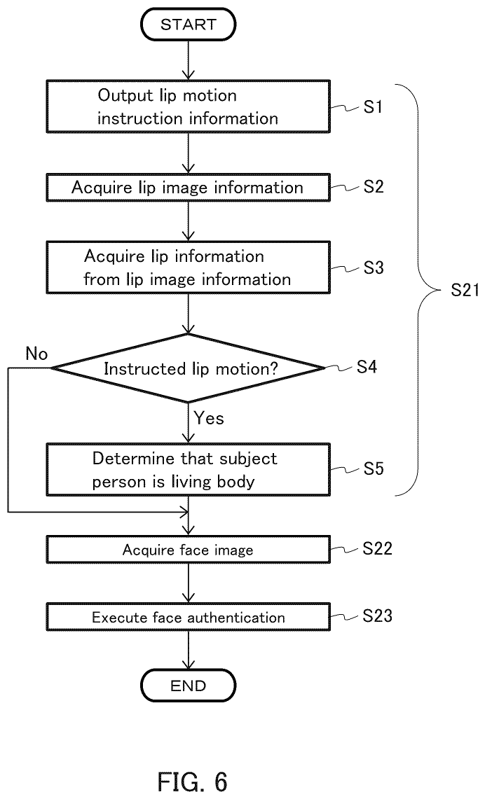

Next, an example of the face authentication method according to the present example embodiment will be described with reference to the flowchart of FIG. 6. The living body detection support method according to the present example embodiment may be carried out as follows using the face authentication apparatus 20 of FIGS. 4 and 5, for example. Note that, the face authentication method according to the present example embodiment is not limited to the use of the face authentication apparatus 20 of FIGS. 4 and 5.

Firstly, the living body detection support part 10 carries out S1 to S5 in the same manner as the processes of S1 to S5 in the first example embodiment (S21, supporting living body detection). Note that, the supporting living body detection is also referred to as biologically detecting, for example.

Next, the face image acquisition part 21 of the face authentication apparatus 20 acquires a face image of an authentication subject person (S22, acquiring a face image). The face image is not particularly limited as long as being an image imaging a face of an authentication subject person, and for example, may be a still image such as a photograph, or a moving image. The face image acquisition part 21 may acquire the image continuously or intermittently, for example. In the latter cases, the image may be acquired at every given time elapsed, or at any timing. The face image acquisition part 21 may acquire the face image by imaging the face of the authentication subject person with the imaging device, which is the input unit 206, for example, but may acquire the face image from an external imaging device by the communication device 208 via the communication line network. When the lip image information acquired in S2 is a face image of the subject person, the lip image information may be acquired as the face image of the authentication subject person. The face image acquisition part 21 may, for example, store the acquired face image in the memory 202 or the storage 204.

Next, the face authentication part 22 of the face authentication apparatus 20 executes the face authentication of the subject person on the basis of the face image of the subject person and authentication criteria information (S23, executing face authentication). Examples of the authentication criteria information include information such as a pre-stored face image and a facial feature quantity of the authentication subject person. The process by the face authentication part 22 is not particularly limited, and for example, can be executed in the same manner as a known face-authentication method.

In the face authentication method according to the present example embodiment, a case in which S22 and S23 are sequentially performed after S21 (S1 to S5) is performed was described as an example, but the present invention is not limited thereto. In the present invention, S21, S22 and S23 may be performed simultaneously or separately, for example. In the latter case, the order to perform is not particularly limited and may be any order. When executing S21 after executing S22 and S23, for example, the biologically detecting in S21 may be performed to an authentication subject person who has passed the face authentication.

Since the face authentication apparatus of the present example embodiment includes a living body detection support part, it can prevent spoofing in face authentication.

### Third Example Embodiment

A first program of the present example embodiment is a program for causing a computer to execute each of the processes in the living body detection support method. Specifically, the program of the present example embodiment is a program for causing a computer to execute an instruction information output process, an image information acquisition process, a lip information acquisition process, a lip motion determination process, and a living body determination process.

The instruction information output process outputs lip motion instruction information, the lip motion instruction information includes information instructing a given type of lip motion, the image information acquisition process acquires lip image information including a mouth of a subject person, the lip information acquisition process acquires lip information of the subject person from the lip image information, the lip motion determination process determines whether the subject person performed the instructed lip motion, on the basis of determination criteria information, the lip motion instruction information, and the lip information of the subject person, the determination criteria information is information in which types of lip motion and lip information for each of the types of lip motion are associated with each other, and the living body determination process determines that the subject person is a living body when it is determined that the subject person performed the instructed lip motion.

The first program of the present example embodiment can also be referred to as a program for causing a computer to function as the instruction information output process, the image information acquisition process, the lip information acquisition process, the lip operation determination process, and the living body determination process.

The first program of the present example embodiment may incorporate the descriptions in the living body detection support apparatus and the living body detection support method according to the present invention. In each of the processes, for example, “process” and “processing” can be interchangeably used. The program of the present example embodiment may be recorded in a computer-readable recording medium, for example. The recording medium is, for example, a non-transitory computer-readable recording medium (storage medium). The recording medium is not particularly limited and examples thereof include a random access memory (RAM), a read only memory (ROM), a hard disk (HD), a flash memory (e.g., SSD (Solid State Drive), a USB flash memory, and a SD/SDHC card), an optical disc (e.g., CD-R/CD-RW, DVD-R/DVD-RW, and BD-R/BD-RE), a magneto-optical disk (MO), and a FLOPPY (registered trademark) disk (FD). The program (may be referred to as a programming product or a program product, for example) of the present example embodiment may be in a form to be distributed from an external computer, for example. The “distribution” may be distribution via a communication line network or distribution via a wired-connected device. The program of the present example embodiment may be executed by being installed in a device where the program is distributed, or may be executed without being installed.

### Fourth Example Embodiment

A second program of the present example embodiment is a program for causing a computer to execute each of the processes in the face authentication method described above. Specifically, the program of the present example embodiment is a program for causing a computer to execute a living body detection process, a face image acquisition process and an authentication process.

The living body detection process is executed by the first program, and executes living body detection of an authentication subject person, the face image acquisition process acquires a face image of the authentication subject person, and the authentication process executes face authentication of the authentication subject person, on the basis of the face image of the authentication subject person and authentication criteria information.

The second program of the present example embodiment can also be referred to as a program for causing a computer to function as the living body detection process, the face image acquisition process, and the authentication process.

The second program of the present example embodiment can incorporate the descriptions in the face authentication apparatus and the face authentication method according to the present invention. In each of the processes, for example, “process” and “processing” can be interchangeably used. The program of the present example embodiment may be recorded in a computer-readable recording medium, for example. The recording medium is, for example, a non-transitory computer-readable recording medium (storage medium). The recording medium is not particularly limited and examples thereof include a random access memory (RAM), a read only memory (ROM), a hard disk (HD), a flash memory (e.g., SSD (Solid State Drive), a USB flash memory, and a SD/SDHC card), an optical disc (e.g., CD-R/CD-RW, DVD-R/DVD-RW, and BD-R/BD-RE), a magneto-optical disk (MO), and a FLOPPY (registered trademark) disk (FD). The program (may be referred to as a programming product or a program product, for example) of the present example embodiment may be in a form to be distributed from an external computer, for example. The “distribution” may be distribution via a communication line network or distribution via a wired-connected device. The program of the present example embodiment may be executed by being installed in a device where the program is distributed, or may be executed without being installed.

While the invention has been particularly shown and described with reference to example embodiments thereof, the invention is not limited to these embodiments. It will be understood by those of ordinary skill in the art that various changes in form and details may be made therein without departing from the spirit and scope of the present invention as defined by the claims.

### SUPPLEMENTARY NOTE

The whole or part of the example embodiments disclosed above can be described as, but not limited to, the following supplementary notes.

### Supplementary Note 1

A living body detection support apparatus, including:
a determination criteria information storage part;
an instruction information output part;
an image information acquisition part;
a lip information acquisition part;
a lip motion determination part; and
a living body determination part, wherein
the determination criteria information storage part stores determination criteria information,
the determination criteria information is information in which types of lip motion and lip information for each of the types of lip motion are associated with each other,
the instruction information output part outputs lip motion instruction information,
the lip motion instruction information includes information instructing a given type of lip motion,
the image information acquisition part acquires lip image information including a mouth of a subject person,
the lip information acquisition part acquires lip information of the subject person from the lip image information,
the lip motion determination part determines whether the subject person performed the instructed lip motion, on the basis of the determination criteria information, the lip motion instruction information, and the lip information of the subject person, and
the living body determination part determines that the subject person is a living body when the lip motion determination part determines that the subject person performed the instructed lip motion.

### Supplementary Note 2

The living body detection support apparatus according to Supplementary Note 1, wherein
the lip information includes lip coordinate information,
the determination criteria information includes information in which types of lip motion and lip coordinate information for each of the types of lip motion are associated with each other,
the lip information acquisition part acquires the lip coordinate information of the subject person from the lip image information as the lip information, and
the lip motion determination part specifies reference lip coordinate information on the basis of the determination criteria information and the lip motion instruction information, and execute matching of the reference lip coordinate information and the lip coordinate information of the subject person to determine whether the subject person performed the instructed lip motion.

### Supplementary Note 3

The living body detection support apparatus according to Supplementary Note 1 or 2, wherein
the type of lip motion includes phonation of a given character,
the determination criteria information includes information in which types of given characters and lip information for each of the types of characters are associated with each other, and
the instruction information output part outputs information instructing the phonation of the given character, as the lip motion instruction information.

### Supplementary Note 4

A face authentication apparatus, including:
a living body detection part;
a face image acquisition part; and
an authentication part, wherein
the living body detection part is the living body detection support apparatus according to any one of Supplementary Notes 1 to 3, and executes living body detection of an authentication subject person,
the face image acquisition part acquires a face image of the authentication subject person, and
the authentication part executes face authentication of the authentication subject person on the basis of the face image of the authentication subject person and authentication criteria information.

### Supplementary Note 5

A living body detection support method, including:
outputting instruction information;
acquiring image information;
acquiring lip information;
determining lip motion; and
determining living body, wherein
the outputting instruction information outputs lip motion instruction information,
the lip motion instruction information includes information instructing a given type of lip motion,
the acquiring image information acquires lip image information including a mouth of a subject person,
the acquiring lip information acquires the lip information of the subject person from the lip image information,
the determining lip motion determines whether the subject person performed the instructed lip motion, on the basis of determination criteria information, the lip motion instruction information, and the lip information of the subject person,
the determination criteria information is information in which types of lip motion and lip information for each of the types of lip motion are associated with each other, and
the determining living body determines that the subject person is a living body, when it is determined that the subject person performed the instructed lip motion.

### Supplementary Note 6

The method according to Supplementary Note 5, wherein
the lip information includes lip coordinate information,
the determination criteria information includes information in which a given type of lip motion and lip coordinate information for each of the types of lip motion are associated with each other,
the acquiring lip information acquires the lip coordinate information of the subject person from the lip image information, as the lip information,
the determining lip motion specifies reference lip coordinate information on the basis of the determination criteria information and the lip motion instruction information, and executes matching of the reference lip coordinate information and the lip coordinate information of the subject person to determine whether the subject person performed the instructed lip motion.

### Supplementary Note 7

The method according to Supplementary Note 5 or 6, wherein
the type of lip motion includes phonation of a given character,
the determination criteria information includes information in which types of given characters and lip information for each of the types of characters are associated with each other,
the outputting instruction information outputs information instructing the phonation of a given character, as the lip motion instruction information.

### Supplementary Note 8

A face authentication method, including:
detecting a living body;
acquiring a face image; and
authenticating, wherein
the detecting a living body is the living detection support method according to any one of Supplementary Notes 5 to 7, and executes living body detection of an authentication subject person,
the acquiring a face image acquires the face image of the authentication subject person, and
the authenticating executes face authentication of the authentication subject person on the basis of the face image of the authentication subject person and authentication criteria information.

### Supplementary Note 9

A program for causing a computer to execute following processes, including:
an instruction information output process;
an image information acquisition process;
a lip information acquisition process;
a lip motion determination process; and
a living body determination process, wherein
the instruction information output process outputs lip motion instruction information,
the lip motion instruction information includes information instructing a given type of lip motion,
the image information acquisition process acquires lip image information including a mouth of a subject person,
the lip information acquisition process acquires lip information of the subject person from the lip image information,
the lip motion determination process determines whether the subject person performed the instructed lip motion, on the basis of determination criteria information, the lip motion instruction information, and the lip information of the subject person,
the determination criteria information is information in which types of lip motion and lip information for each of the types of lip motion are associated with each other, and
the living body determination process determines that the subject person is a living body when it is determined that the subject person performed the instructed lip motion.

### Supplementary Note 10

The program according to Supplementary Note 9, wherein
the lip information includes lip coordinate information,
the determination criteria information includes information in which types of lip motion and lip coordinate information for each of the types of lip motion are associated with each other,
the lip information acquisition process acquires lip coordinate information of the subject person from the lip image information, as the lip information, and
the lip motion determination process specifies reference lip coordinate information on the basis of the determination criteria information and the lip motion instruction information, and executes matching of the reference lip coordinate information and the lip coordinate information of the subject person to determine whether the subject person performed the instructed lip motion.

### Supplementary Note 11

The program according to Supplementary Note 9 or 10, wherein
the type of lip motion includes phonation of a given character,
the determination criteria information includes information in which types of given characters and lip information for each of the types of characters are associated with each other,
the instruction information output process outputs information instructing the phonation of a given character, as the lip motion instruction information.

### Supplementary Note 12

A program for causing a computer to execute following processes, including:
a living body detection process;
a face image acquisition process; and
an authentication process, wherein
the living body detection process is executed by the program according to any one of Supplementary Notes 9 to 11, and executes living body detection of an authentication subject person,
the face image acquisition process acquires a face image of the authentication subject person, and
the authentication process executes face authentication of the authentication subject person, on the basis of the face image of the authentication subject person and authentication criteria information.

### Supplementary Note 13

A computer-readable recording medium recorded with the program according to any one of Supplementary Notes 9 to 12.

### INDUSTRIAL APPLICABILITY

The present invention can achieve living body detection of a subject person, by for example, performing matching of randomly decided lip motion and lip motion performed by the subject person. Thus, the present invention is widely useful in various fields in which living body detection is necessary.

### REFERENCE SIGNS LIST

10: living body detection support apparatus
11: determination criteria information storage part
111: determination criteria information
12: instruction information output part
13: image information acquisition part
14: lip information acquisition part
15: lip motion determination part
16: living body determination part
101: CPU
102: memory
103: bus
104: storage
105: program
106: input unit
107: output unit
108: communication device
20: face authentication apparatus
21: face image acquisition part
22: face authentication part
201: CPU
202: memory
203: bus
204: storage
205: program
206: input unit
207: display
208: communication device

What is claimed is:

1. An apparatus for living body detection support, comprising: at least one processor and at least one storage, wherein
the storage is configured to: store determination criteria information, wherein,
the determination criteria information is information in which types of lip motion and lip information for each of the types of lip motion are associated with each other,
the processor is configured to:
output lip motion instruction information, wherein
the lip motion instruction information includes information instructing a given type of lip motion,
acquire lip image information including a mouth of a subject person,
acquire lip information of the subject person from the lip image information,
determine whether the subject person performed the instructed lip motion, on the basis of the determination criteria information, the lip motion instruction information, and the lip information of the subject person, and
determine that the subject person is a living body when it is determined that the subject person performed the instructed lip motion.

2. The apparatus according to claim 1, wherein
the lip information includes lip coordinate information,
the determination criteria information includes information in which types of lip motion and lip coordinate information for each of the types of lip motion are associated with each other,
the processor is configured to:
acquire the lip coordinate information of the subject person from the lip image information as the lip information, and
specify reference lip coordinate information on the basis of the determination criteria information and the lip motion instruction information, and execute matching of the reference lip coordinate information and the lip coordinate information of the subject person to determine whether the subject person performed the instructed lip motion.

3. The apparatus according to claim 1, wherein
the type of lip motion includes phonation of a given character,
the determination criteria information includes information in which types of given characters and lip information for each of the types of characters are associated with each other, and
the processor is configured to output information instructing the phonation of the given character, as the lip motion instruction information.

4. An apparatus for face authentication, comprising at least one processor and
the apparatus according to claim 1, the apparatus is configured to execute living body detection of an authentication subject person,
the processor is configured to:
acquire a face image of the authentication subject person, and
execute face authentication of the authentication subject person on the basis of the face image of the authentication subject person and authentication criteria information.

5. A computer-implemented method for a living body detection support, comprising:
outputting lip motion instruction information,
the lip motion instruction information includes information instructing a given type of lip motion,
acquiring lip image information including a mouth of a subject person,
acquiring the lip information of the subject person from the lip image information,
determining whether the subject person performed the instructed lip motion, on the basis of determination criteria information, the lip motion instruction information, and the lip information of the subject person,
the determination criteria information is information in which types of lip motion and lip information for each of the types of lip motion are associated with each other, and
determining that the subject person is a living body, when it is determined that the subject person performed the instructed lip motion.

6. The method according to claim 5, wherein
the lip information includes lip coordinate information,
the determination criteria information includes information in which a given type of lip motion and lip coordinate information for each of the types of lip motion are associated with each other,
the method comprising:
acquiring the lip coordinate information of the subject person from the lip image information, as the lip information, and
determining specifies reference lip coordinate information on the basis of the determination criteria information and the lip motion instruction information, and executes matching of the reference lip coordinate information and the lip coordinate information of the subject person to determine whether the subject person performed the instructed lip motion.

7. The method according to claim 5, wherein
the type of lip motion includes phonation of a given character,
the determination criteria information includes information in which types of given characters and lip information for each of the types of characters are associated with each other,
the method comprising outputting information instructing the phonation of a given character, as the lip motion instruction information.

8. A computer-implemented method for a face authentication, comprising:
the method according to claim 5;
acquiring the face image of the authentication subject person, and
authenticating face authentication of the authentication subject person on the basis of the face image of the authentication subject person and authentication criteria information.

9-12. (canceled)

13. A non-transitory computer-readable recording medium recorded with a program, wherein
the program causes a computer to execute a method for a living body detection support, wherein,
the method comprising:
outputting lip motion instruction information,
the lip motion instruction information includes information instructing a given type of lip motion,
acquiring lip image information including a mouth of a subject person,
acquiring the lip information of the subject person from the lip image information,
determining whether the subject person performed the instructed lip motion, on the basis of determination criteria information, the lip motion instruction information, and the lip information of the subject person,
the determination criteria information is information in which types of lip motion and lip information for each of the types of lip motion are associated with each other, and
determining that the subject person is a living body, when it is determined that the subject person performed the instructed lip motion.

14. The recording medium according to claim 13, wherein
the lip information includes lip coordinate information,
the determination criteria information includes information in which a given type of lip motion and lip coordinate information for each of the types of lip motion are associated with each other,
the method comprising:
acquiring the lip coordinate information of the subject person from the lip image information, as the lip information, and
determining specifies reference lip coordinate information on the basis of the determination criteria information and the lip motion instruction information, and executes matching of the reference lip coordinate information and the lip coordinate information of the subject person to determine whether the subject person performed the instructed lip motion.

15. The recording medium according to claim 13, wherein
the type of lip motion includes phonation of a given character,
the determination criteria information includes information in which types of given characters and lip information for each of the types of characters are associated with each other,
the method comprising outputting information instructing the phonation of a given character, as the lip motion instruction information.

16. A non-transitory computer-readable recording medium recorded with a program, wherein
the program causes a computer to execute a method for a face authentication, wherein,
the method comprising:
the method according to claim 5;
acquiring the face image of the authentication subject person, and
authenticating face authentication of the authentication subject person on the basis of the face image of the authentication subject person and authentication criteria information.
